(19) United States
(12) Patent Application Publication
(10) Pub. No.: US 2017/0328921 A1
(43) Pub. Date: Nov. 16, 2017

(54) METHODS FOR DETECTING HORMONES AND OTHER ANALYTES

(71) Applicant: Sanis Biomedical, LLC, Edmond, OK (US)

(72) Inventor: Kris Franklin, Mandeville, LA (US)

(73) Assignee: Sanis Biomedical, LLC, Edmond, OK (US)

(21) Appl. No.: 15/612,255

(22) Filed: Jun. 2, 2017

Related U.S. Application Data
(60) Provisional application No. 62/330,588, filed on May 2, 2016.

(51) Int. Cl. G01N 33/74; G01N 30/14; G01N 30/72

(52) U.S. Cl. CPC G01N 33/743; G01N 30/7233; G01N 30/14; G01N 2560/00

(57) ABSTRACT

The present application relates to methods for determining the concentration of one or more hormones in a sample by liquid chromatography-tandem mass spectrometry (LC-MS/MS).

### CROSS-REFERENCE TO RELATED APPLICATIONS

This application claims the benefit of 35 U.S.C. §119 based on the priority of U.S. Provisional Patent Application No. 62/330,588 filed May 2, 2016, which is hereby incorporated by reference.

### FIELD

The present application relates to methods for detecting analytes in a sample. In particular, the present application relates to methods for determining the concentration of one or more analytes such as hormones in a small volume sample by liquid chromatography-tandem mass spectrometry (LC-MS/MS).

### BACKGROUND

Measuring many different substances such as hormones in a sample is desirable in medicine and many other biologically related fields. Conventional approaches can include multi-analyte immunological assays, where different labels are used for different analytes. The number of analytes that can be assessed can be limited by the number of labels available.

High performance liquid chromatography-mass spectrometry (LC-MS) is a technique that combines high performance liquid chromatography (HPLC) with mass spectrometry. In HPLC, a sample is forced by a liquid (the mobile phase) at high pressure through a column that is packed with a stationary phase so as to physically separate the components of the sample. Mass spectrometry is a technique that measures the mass-to-charge ratio (m/z) of charged particles.

In general, mass spectrometers contain four fundamental components: a sample inlet device that mediates the transition of a solid or liquid specimen into the gaseous phase; an ionization device that ionizes the vaporized samples; an ion path that transitions ions from the source (which is close to atmospheric pressure) to a mass analyzer (which is under high vacuum) and moves them towards a detector while separating them from each other based on their m/z; and an ion detector to detect and quantify ions (Grebe & Singh, 2011).

Double quadrupole mass spectrometers comprise two quadrupoles. In a triple quadrupole mass spectrometer (a type of tandem mass spectrometer, MS/MS), three quadrupoles are arranged in a linear sequence and each quadrupole uses a combination of radio frequency and direct current potentials in order to select for masses (de Hoffmann, 1995). The first quadrupole (Q1) serves as both a mass analyzer and a filter for precursor ions traveling through the mass spectrometer. The second quadrupole is a collision chamber where ion fragmentation occurs (from parent to daughter ion). After fragmentation occurs, the ions are then focused into the third quadrupole (Q3) where they undergo additional filtering before hitting the detector.

In multiple reaction monitoring (MRM), a specific m/z is selected in the first mass-filtering device (Q1), the collision chamber is filled with a collision gas to fragment the selected m/z, and one specific m/z is selected in in the second mass-filtering device (Q3) for detection (Grebe & Singh, 2011).

Tandem mass spectrometry is useful for the measurement of small molecules such as steroids, drugs and intermediary metabolites as well as many other analytes.

Interference by substances that co-elute in preparatory columns and by epimers and structural isomers can pose problems in multi-analyte testing.

### Instrument Calibration and Tuning

In order to ensure the accuracy of measurements made by mass spectrometers, it is recognized as good practice for the machine to be calibrated and tuned (Barwick et al., 2006). Calibration of the machine involves the measurement of a compound that is a “well-defined standard” (Ho et al., 2003). This standard has a known mass-to-charge (m/z) ratio, allowing for the technician to gauge the accuracy of the machine.

Tuning is a process that, in theory, optimizes the machine's sensitivity or response. For example, tuning on instruments is typically accomplished using a vendor-provided solution, as well as an automatic/software directed procedure. The tuning process involves altering parameters (e.g. interface voltages, etc.) while measuring a standard and monitoring the change in response. Once the machine finds the parameters resulting in the best response, it is saved as a file and used in subsequent measurements. Traditionally, once a machine is tuned, the tune file's parameters are not altered and are used in all subsequent measurements (Barwick et al., 2006).

### DL Temperature Variation

In many applications, the desolvation line (DL) temperature is increased in order to produce better ionization results. Many known methods, including those that focus on hormones, use DL temperatures at or above 200° C. (Sawant et al., Nguyen et al., 2010; Gervasomi et al., 2014), the rationale being that using higher temperatures leads to an increase in solvent evaporation, and less solvent reaching the optics of the instrument. It would therefore follow there would be significantly less noise expected due to solvent.

### Additions to Sample Diluent

Hormones are non-polar molecules with few easily ionizable functional groups. In many applications, weak acids e.g. formic acid have been used as an addition to the sample diluent.

### Compound Optimization

All high performance liquid chromatography-tandem mass spectrometry (LC-MS/MS) platforms have some type of recommended optimization protocol. These steps are standardized and have voltage guidance for optimizing a compound. The MRM optimization is the cornerstone for being able to actively target a compound for quantitation.

### SUMMARY

The present application relates to a method for determining the concentration of one or more analytes in a sample by liquid chromatography-tandem mass spectrometry (LC-MS/MS), the method comprising:
subjecting at least a portion of the sample to LC-MS/MS under conditions to generate a precursor ion for each of the one or more analytes and to generate one or more product ions from each of the precursor ions;
detecting the amount of the one or more product ions; and
relating the amount of the one or more detected product ions to the concentration of the one or more analytes in the sample,
   wherein
the conditions for the liquid chromatography comprise a binary gradient using a first mobile phase and a second mobile phase, both mobile phases comprising ammonium fluoride;
the conditions for the tandem mass spectrometry comprise a desolvation line temperature of less than 200° C.; and/or
the conditions for the tandem mass spectrometry comprise an interface voltage of less than about 4 kV.

In an embodiment, the sample is a biological sample, optionally wherein the sample is a urine sample or a blood sample optionally selected from a whole blood sample, a serum sample and a plasma sample. In another embodiment, the blood sample is a capillary blood sample. In a further embodiment, the blood sample is a venous blood sample.

In a further embodiment, the sample is a serum sample and, prior to being subjected to LC-MS/MS, is prepared by a method comprising:
loading the sample onto a supported liquid extraction (SLE) column and eluting with an elution solvent under conditions to collect the one or more analytes in the elution solvent on a sample plate;
drying under conditions to remove the elution solvent; and
reconstituting the sample in a sample diluent comprising ammonium fluoride.

In another embodiment, about 150 μL of the serum sample is loaded onto the SLE column. In a further embodiment, the sample diluent consists essentially of about 0.25 mM to about 2 mM NH₄F in a methanol-water solution. It is an embodiment that the sample diluent consists essentially of about 0.5 mm NH₄F in in about 50:50 v/v methanol:water. In another embodiment of the present application, the sample plate is a silated 96-well deep well plate.

In another embodiment, the sample is a whole blood sample and the method further comprises preparing a serum sample from the whole blood sample by a method comprising centrifuging the whole blood sample under conditions to separate the serum from blood cells, optionally by removing clotted blood cells.

In an embodiment, the first mobile phase consists essentially of an aqueous solution of from about 0.25 mM to about 2 mM, optionally about 0.5 mM ammonium fluoride. In another embodiment of the present application, the second mobile phase consists essentially of a methanolic solution of from about 0.25 mM to about 2 mM, optionally about 0.5 mM ammonium fluoride.

In an embodiment, the conditions for the high performance liquid chromatography comprise a total flow rate of from about 0.50 mL/minute to about 1.0 mL/minute or about 0.70 mL/minute.

In an embodiment, the one or more analytes are steroid hormones. In another embodiment, the one or more analytes are corticosteroids, sex steroids or combinations thereof. In a further embodiment, the one or more hormones are selected from aldosterone, androstenedione, corticosterone, cortisol, cortisone, 21-deoxycortisol, 11-deoxycortisol, dehydroepiandrosterone (DHEA), 11-deoxycorticosterone, estrone, 17-β-estradiol, estriol, 17-α-hydroxyprogesterone, progesterone, testosterone and dihydrotestosterone.

In an embodiment, one hormone is DHEA, the precursor ion has a mass-to-charge ratio (m/z) of 271 and the product ion has a m/z of 213.

In another embodiment, one hormone is aldosterone, the precursor ion has a m/z of 361 and the product ion has a m/z of 315.2, and optionally the conditions for tandem mass spectrometry comprise running in positive ion mode.

In an embodiment, the desolvation line temperature is about 140° C. In another embodiment, the interface voltage is from about 2 kV to about 3 kV, optionally about 2 kV or about 3 kV.

In an embodiment, the liquid chromatography comprises high performance liquid chromatography or ultra-high performance liquid chromatography. In another embodiment, the liquid chromatography is high performance liquid chromatography. In a further embodiment, the tandem mass spectrometer is a triple quadrupole mass spectrometer.

In an embodiment, the samples are loaded onto the LC-MS/MS on a sample plate. For example, the sample plate containing the reconstituted samples is loaded onto the LC-MS/MS. In another embodiment, the sample plate further comprises one or more calibrators, quality control samples and/or blanks.

In an embodiment, the method further comprises adding an internal standard to the sample, and, if present, to the one or more calibrators, quality control samples and a portion of the blanks,

In an embodiment, about 10 μL to about 30 μL or about 20 μL of the reconstituted sample is loaded onto the LC-MS/MS.

Other features and advantages of the present application will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

### BRIEF DESCRIPTION OF THE DRAWINGS

The present application will now be described in greater detail with reference to the drawings in which:

FIG. 1 is a schematic showing an exemplary sample plate set-up for use in the methods of the present application.

FIG. 2 shows changes in response as a function of interface voltage (2 kV (the control), 3 kV, 4 kV (the tune voltage), or 5 kV) for estriol (E3), cortisol, cortisone, 21-deoxycortisol, 17-β-estradiol (E2), aldosterone, 11-deoxycortisol, corticosterone, estrone (E1), dehydroepiandrosterone (DHEA), testosterone, 17-α-hydroxyprogesterone, 11-deoxycorticosterone, androstenedione, progesterone and dihydrotestosterone (DHT) (from left to right).

FIG. 3 shows changes in response as a function of desolvation line (DL) temperature (140, 200, 250 or 300° C.) for estriol, cortisol, cortisone, 21-deoxycortisol, 17-β-estradiol (E2), aldosterone, 11-deoxycortisol, corticosterone, estrone, DHEA, testosterone, 17-α-hydroxyprogesterone, 11-deoxycorticosterone, androstenedione, progesterone and DHT (from left to right).

FIG. 4 shows an exemplary precursor ion scan for DHEA according to an embodiment of the methods of the present application.

FIG. 5 shows an exemplary chromatogram (top panel) and multiple reaction monitoring (MRM; bottom panel) for DHEA according to an embodiment of the methods of the present application.

FIG. 6 shows an exemplary precursor ion scan for aldosterone according to an embodiment of the methods of the present application.

FIG. 7 shows an exemplary chromatogram (top panel) and multiple reaction monitoring (MRM; bottom panel) for DHEA according to an embodiment of the methods of the present application.

FIG. 8 shows exemplary chromatograms for (A) aldosterone; (B) cortisone; (C) cortisol; (D) 11-deoxycorticosterone; (E) 17-α-hydroxyprogesterone; (F) 21-deoxycortisol; (G) 11-deoxycortisol; (H) corticosterone; (I) estrone; (J) estradiol; (K) androstenedione; (L) estriol; (M) testosterone; (N) DHEA; and (O) progesterone.

FIG. 9 shows an exemplary full chromatogram for a method of detecting hormones according to an embodiment of the present application. The intensity of the signal is shown as a function of the time in minutes.

### DETAILED DESCRIPTION

I. Definitions

The definitions and embodiments described in this and other sections are intended to be applicable to other embodiments herein described for which they are suitable as would be understood by a person skilled in the art. For example, in the below passages, different aspects of the invention are defined in more detail. Each aspect so defined can be combined with any other aspect or aspects unless clearly indicated to the contrary. For example, any feature indicated as being preferred or advantageous can be combined with any other feature or features indicated as being preferred or advantageous.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% or at least ±3% of the modified term if this deviation would not negate the meaning of the word it modifies.

More specifically, the term “about” means plus or minus 0.1 to 50%, 5-50%, 10-40%, 10-20%, 10%-15%, preferably 5-10%, most preferably 5% or 3% of the number to which reference is being made.

The term “and/or” as used herein means that the listed items are present, or used, individually or in combination. In effect, this term means that “at least one of” or “one or more” of the listed items is used or present.

As used in this application, the singular forms “a”, “an” and “the” include plural references unless the content clearly dictates otherwise. For example, an embodiment including “a product ion” should be understood to present certain aspects with one product ion or two or more additional product ions. It should also be noted that the term “or” is generally employed in its sense including “and/or” unless the content clearly dictates otherwise.

In embodiments comprising an “additional” or “second” component, such as an additional or second product ion, the second component as used herein is different from the other components or first component. A “third” component is different from the other, first, and second components, and further enumerated or “additional” components are similarly different.

The recitation of numerical ranges by endpoints herein includes all numbers and fractions subsumed within that range (e.g. 1 to 5 includes 1, 1.5, 2, 2,75, 3, 3.90, 4 and 5). It is also to be understood that all numbers and fractions thereof are presumed to be modified by the term “about”.

The abbreviation “m/z” as used herein refers to the mass:charge ratio of charged particles which is measured by the mass spectrometer.

II. Methods

An aspect of the application includes a method for determining the concentration of one or more analytes in a sample by liquid chromatography-tandem mass spectrometry (LC-MS/MS), the method comprising:
subjecting at least a portion of the sample to LC-MS/MS under conditions to generate a precursor ion for each of the one or more analytes and to generate one or more product ions from each of the precursor ions;
detecting the amount of the one or more product ions; and
relating the amount of the one or more detected product ions to the concentration of the one or more analytes in the sample,
   wherein
the conditions for the liquid chromatography comprise a binary gradient using a first mobile phase and a second mobile phase, both mobile phases comprising ammonium fluoride;
the conditions for the tandem mass spectrometry comprise a desolvation line temperature of less than 200° C.; and/or
the conditions for the tandem mass spectrometry comprise an interface voltage of less than about 4 kV.

The sample can be any suitable sample. In an embodiment, the sample is a biological sample. For example, the sample can be a urine sample or a blood sample. In another embodiment, the sample is a blood sample. In a further embodiment, the sample is selected from the group consisting of a whole blood sample, a serum sample and a plasma sample. It is an embodiment that the sample is a whole blood sample. In another embodiment of the present application, the sample is a serum sample. The methods of the present application can use very small portions of the sample for analysis. Accordingly, in another embodiment, the sample is a capillary blood sample.

In an embodiment, the sample volume is about 350 microliters for whole blood and 175 microliters for serum. A person skilled in the art would readily understand that whole blood samples are typically kept refrigerated prior to use in the methods of the present application. In an embodiment, whole blood samples are used within 3 days (e.g. within 72 his) after capillary draw, unfrozen serum samples are used when 7 or less than 7 days old and frozen serum samples are used when 14 or less than 14 days old.

In an embodiment, the sample is a serum sample and, prior to being subjected to LC-MS/MS, is prepared by a method comprising:
loading the sample onto a supported liquid extraction (SLE) column and eluting with an elution solvent under conditions to collect the one or more analytes in the elution solvent on a sample plate;
drying under conditions to remove the elution solvent; and
reconstituting the sample in a sample diluent comprising ammonium fluoride.

In an embodiment, the sample further comprises an internal standard. In another embodiment of the present application, a desired amount, optionally about 200 μL of the internal standard is added to the well containing the sample prior to loading on the SLE.

In an embodiment, about 10 μL to about 30 μL or about 20 μL of the reconstituted sample is loaded onto the LC-MS/MS.

The conditions to collect the one or more analytes in the elution solvent on the sample plate can be any suitable conditions. For example, the SLE column can be any suitable SLE column and the elution solvent can be any suitable elution solvent, the selection of both of which can be readily made by a person skilled in the art. In the examples of the present application, it was found that using non-polar organic solvents produced a better signal-to-noise ratio on the mass spectrometer. Accordingly, in an embodiment, the elution solvent comprises, consists essentially of or consists of any suitable non-polar solvent or a mixture of non-polar solvents. In another embodiment, the elution solvent comprises, consists essentially of or consists of a mixture of hexane and methyl t-butyl ether. In a further embodiment, the elution solvent comprises, consists essentially of or consists of from about 50:50 v/v to about 70:30 v/v or about 60:40 v/v mixture of hexane and methyl t-butyl ether. In an embodiment, the method comprises two sequential elutions with the elution solvent. In another embodiment of the present application, from about 125 μL to about 175 μL or about 150 μL of the serum sample is loaded onto the SLE column.

The conditions to remove the elution solvent can be any suitable conditions. In an embodiment, the conditions comprise drying under an inert gas such as nitrogen at a temperature of from about 30° C. to about a0° C. or about 40° C.

The solvent in the sample diluent comprising ammonium fluoride can be any suitable solvent or mixtures of solvents, the selection of which can be made by a person skilled in the art. For example, it would be appreciated by a person skilled in the art that the solvent(s) are selected so that the ammonium fluoride is soluble therein. In an embodiment, at least a portion of the solvent in the sample diluent is water. In another embodiment, the sample diluent comprises, consists essentially of or consists of ammonium fluoride in a methanol-water solution. In a further embodiment, the concentration of ammonium fluoride (NH₄F) in the sample diluent is from about 0.25 mM to about 2 mM or about 0.5 mM. For example, the sample diluent can comprise, consist essentially of or consist of about 0.25 mM to about 2 mM NH₄F in a methanol-water solution, optionally wherein the methanol-water solution is a from about 40:60 v/v to about 60:40 v/v or about 50:50 v/v mixture of methanol:water.

In another embodiment, the sample plate is a silated sample plate, optionally wherein the sample plate is a silated 96-well deep well plate.

In an embodiment, the sample is a whole blood sample and the method further comprises preparing a serum sample from the whole blood sample by a method comprising centrifuging the whole blood sample under conditions to separate the serum from blood cells, optionally by removing clotted blood cells. It will be appreciated by a person skilled in the art that such serum samples can then, prior to being subjected to LC-MS/MS, be prepared as described herein for samples which are serum samples. The conditions to separate the serum from the blood cells can be any suitable conditions, the selection of which can be made by a person skilled in the art. For example, in an embodiment, the conditions comprise centrifuging at a temperature of about 4° C. at a setting of about 5000 rcf for a time until there is adequate separation of the serum from the blood cells, for example about 10 minutes.

In an embodiment, the first mobile phase comprises, consists essentially of or consists of an aqueous solution of from about 0.25 mM to about 2 mM, optionally about 0.5 mM ammonium fluoride.

In an embodiment, the second mobile phase comprises, consists essentially of or consists of a methanolic solution of from about 0.25 mM to about 2 mM, optionally about 0.5 mM ammonium fluoride.

In an embodiment, the conditions for the high performance liquid chromatography comprise a total flow rate of from about 0.50 mL/minute to about 1.0 mL/minute or about 0.70 mL/minute. In another embodiment, the ratio of the second mobile phase to the first mobile phase is about 60:40 v/v at 0.01 minutes, about 75:25 v/v at about 2.00 minutes, about 75:25 v/v at about 4.00 minutes, about 95:5 v/v at about 5.00 minutes, about 95:5 v/v at about 8.00 minutes and/or about 60:40 v/v at about 8.10 minutes and until the flow is stopped. In an embodiment, the flow is stopped at about 9.20 minutes.

The analyte can be any small molecule (e.g. a molecule having a molecular weight of less than about 1000 or about 900 daltons) organic species that is hydrophobic and has non-ionizable functional groups. For example, the analyte can be any steroid hormone, including for example steroid hormone metabolites and synthetic intermediates as well as structurally related compounds, such as therapeutically administered steroid hormones (e.g. those administered for birth control, performance enhancement or any other prescription steroids). The analyte can also, for example, be a secosteroid (e.g. vitamin D compounds such as cholecalciferol or ergocalciferol); a tocopherol or tocotrienol (e.g. vitamin E compounds); a terpenoid (e.g. a carotene such as beta-carotene); or other fat-soluble vitamins such as the vitamin K family. In an embodiment, the one or more analytes are steroid hormones. In another embodiment, the one or more analytes are corticosteroids, sex steroids or combinations thereof. In another embodiment of the present application, the one or more hormones are selected from aldosterone, androstenedione, corticosterone, cortisol, cortisone, 21-deoxycortisol, 11-deoxycortisol, dehydroepiandrosterone (DHEA), 11-deoxycorticosterone, estrone, 17-β-estradiol, estriol, 17-α-hydroxyprogesterone, progesterone, testosterone, dihydrotestosterone and pregnenolone. In a further embodiment, the one or more hormones are selected from aldosterone, androstenedione, corticosterone, cortisol, cortisone, 21-deoxycortisol, 11 deoxycortisol, dehydroepiandrosterone (DHEA), 11-deoxycorticosterone, estrone, 17-β-estradiol, estriol, 17-α-hydroxyprogesterone, progesterone, testosterone and dihydrotestosterone. For example, the methods of the present application can advantageously be used in a multiplex assay.

A person skilled in the art can readily relate the amount of the one or more detected product ions to the concentration of the one or more analytes in the sample, for example, using standard means and methods for calculation.

In an embodiment, the method determines a concentration of 11-deoxycorticosterone of from about 0.156 to about 5 ng/mL; a concentration of 11-deoxycortisol of from about 0.234 to about 7.5 ng/mL; a concentration of 17-α-hydroxyprogesterone of from about 0.313 ng/mL to about 10 ng/mL; a concentration of 17-β-estradiol of from about 0.09 to about 5 ng/mL; a concentration of 21-deoxycortisol of from about 0.313 to about 10 ng/mL; a concentration of aldosterone of from about 0.234 to about 7.5 ng/mL; a concentration of androstenedione of from about 0.234 to about 7.5 ng/mL; a concentration of corticosterone of from about 0.234 to about 7.5 ng/mL; a concentration of cortisol of from about 0.313 to about 250 ng/mL; a concentration of cortisone of from about 0.313 to about 60 ng/mL; a concentration of DHEA of from about 1 to about 40 ng/mL; a concentration of estriol of from about 0.156 to about 5 ng/mL; a concentration of estrone of from about 0.313 to about 10 ng/mL; a concentration of progesterone of from about 0.156 to about 25 ng/mL; and/or a concentration of testosterone of from about 0.070 to about 10 ng/mL in the sample.

In an embodiment, one hormone is DHEA, the precursor ion has a mass-to-charge ratio (m/z) of 271 and the product ion has a m/z of 213.

In another embodiment, one hormone is aldosterone, the precursor ion has a m/z of 361 and the product ion has a m/z of 315.2. In a further embodiment, the conditions for tandem mass spectrometry when one hormone is aldosterone comprise running in positive ion mode.

In an embodiment, the desolvation line temperature is from about 120° C. to about 180° C. or about 140° C.

In an embodiment, the interface voltage is from about 2 kV to about 3 kV. In another embodiment, the interface voltage is about 2 kV. In a further embodiment, the interface voltage is about 3 kV.

The liquid chromatography can be any suitable liquid chromatography, the selection of which can be made by a person skilled in the art. For example, the liquid chromatography can comprise high performance liquid chromatography or ultra-high performance liquid chromatography, optionally the liquid chromatography is high performance liquid chromatography.

The tandem mass spectrometer can be any suitable tandem mass spectrometer, the selection of which can be made by a person skilled in the art. For example, the methods of the present application can use a double quadrupole mass spectrometer or a triple quadrupole mass spectrometer. In an embodiment, the tandem mass spectrometer is a triple quadrupole mass spectrometer.

The samples can be loaded onto the LC-MS/MS using any suitable means, the selection of which can be made by a person skilled in the art. In an embodiment, the samples are loaded onto the LC-MS/MS on a sample plate, for example, 96-well deep well plate, optionally wherein the sample plate is silated. For example, in an embodiment, the sample plate containing the reconstituted samples is loaded onto the LC-MS/MS. It will be appreciated by a person skilled in the art that the sample plate can optionally also comprise, in addition to one or more samples, one or more calibrators, quality control samples and/or blanks.

In an embodiment, the sample plate comprises a series of calibrators, each of which contains a different level of the one or more analytes, optionally prepared by a method comprising serial dilution of a stock solution. In another embodiment, the sample plate comprises a series of six calibrators, each of which contains a different level of the one or more analytes, optionally prepared by a method comprising serial dilution of a stock solution.

In an embodiment, prior to being subjected to LC-MS/MS, the calibrators are prepared by a method comprising:
adding a desired amount, optionally, about 135 μL of a suitable matrix substitute, optionally charcoal stripped fetal bovine serum to a well, optionally in a well of a 96-well deep well plate;
adding a desired amount, optionally, about 15 μL of the desired calibrator to the well;
loading the matrix substitute and calibrator onto an SLE column and eluting with an elution solvent onto a sample plate;
drying under conditions to remove the elution solvent; and
reconstituting the calibrator in a sample diluent comprising ammonium fluoride,

In an embodiment, the calibrators further comprise an internal standard. In another embodiment of the present application, a desired amount, optionally about 200 μL of the internal standard is added to the well containing the matrix substitute and calibrator prior to loading on the SLE.

In an embodiment, the sample plate comprises one or more, optionally at least two quality control samples. A person skilled in the art would readily be able to select a suitable quality control sample. For example, suitable standards for quality control samples are commercially available such as the UTAK controls used in the examples of the present application. In an embodiment, the sample plate comprises two quality control samples, each of which comprise different levels of the one or more analytes.

In an embodiment, prior to being subjected to LC-MS/MS, the quality control samples are prepared by a method comprising:
adding a desired amount of a suitable matrix substitute, optionally charcoal stripped fetal bovine serum to a well, optionally in a well of a 96-well deep well plate:
adding a desired amount of the desired quality control to the well;
loading the matrix substitute and quality control onto an SLE column and eluting with an elution solvent onto a sample plate;
drying under conditions to remove the elution solvent; and
reconstituting the quality control sample in a sample diluent comprising ammonium fluoride.

In an embodiment, the quality control sample further comprises an internal standard. In another embodiment of the present application, a desired amount, optionally about 200 μL of the internal standard is added to the well containing the matrix substitute and quality control prior to loading on the SLE.

In an embodiment, the sample plate comprises one or more, optionally at least two blanks. In an embodiment, at least one blank is a double blank which is prepared from a suitable matrix substitute, for example, charcoal stripped fetal bovine serum. In another embodiment, at least one blank is a matrix blank which is prepared from a suitable matrix substitute, for example, charcoal stripped fetal bovine serum and an internal standard.

In an embodiment, prior to being subjected to LC-MS/MS, the double blank is prepared by a method comprising:
adding a desired amount, optionally about 150 μL of a suitable matrix substitute, optionally charcoal stripped fetal bovine serum to a well, optionally in a well of a 96-well deep well plate;
loading the matrix substitute onto an SLE column and eluting with an elution solvent onto a sample plate;
drying under conditions to remove the elution solvent; and
reconstituting the double blank in a sample diluent comprising ammonium fluoride.

In an embodiment, prior to being subjected to LC-MS/MS, the matrix blank is prepared by a method comprising:
adding a desired amount, optionally about 150 μL of a suitable matrix substitute, optionally charcoal stripped fetal bovine serum to a well, optionally in a well of a 96-well deep well plate;
adding a desired amount, optionally, about 200 μL of an internal standard to the well;
loading the matrix substitute onto an SLE column and eluting with an elution solvent onto a sample plate;
drying under conditions to remove the elution solvent; and
reconstituting the matrix blank in a sample diluent comprising ammonium fluoride.

It will be appreciated by a person skilled in the art that the methods for preparing the sample and optionally the calibrators, quality control samples and/or blanks are carried out simultaneously; i.e. the desired reagents are each added to their respective wells, and then are loaded onto the SLE column.

The internal standard comprises any suitable isotopically labeled analytes that correspond to the one or more analytes of interest. In an embodiment, the isotopic label is deuterium or ¹³C. However, any non-radioactive isotopic label can be used as an internal standard so long as it possesses a minimum mass difference of M+3 from the analyte of interest.

The following non-limiting examples are illustrative of the present application:

### EXAMPLES

### General Materials and Methods for the Examples

### (a) Sample Suitability

Patient specimens were first analyzed for their suitability for the test. Volumes used for the test were 350 microliters for whole blood and 150 microliters for serum. Whole blood samples were kept refrigerated prior to receipt. Whole blood samples were received typically within 3 days after capillary draw, and serum samples were typically less than 7 days old, unless frozen (up to 14 days).

### (b) General Sample Preparation Procedure

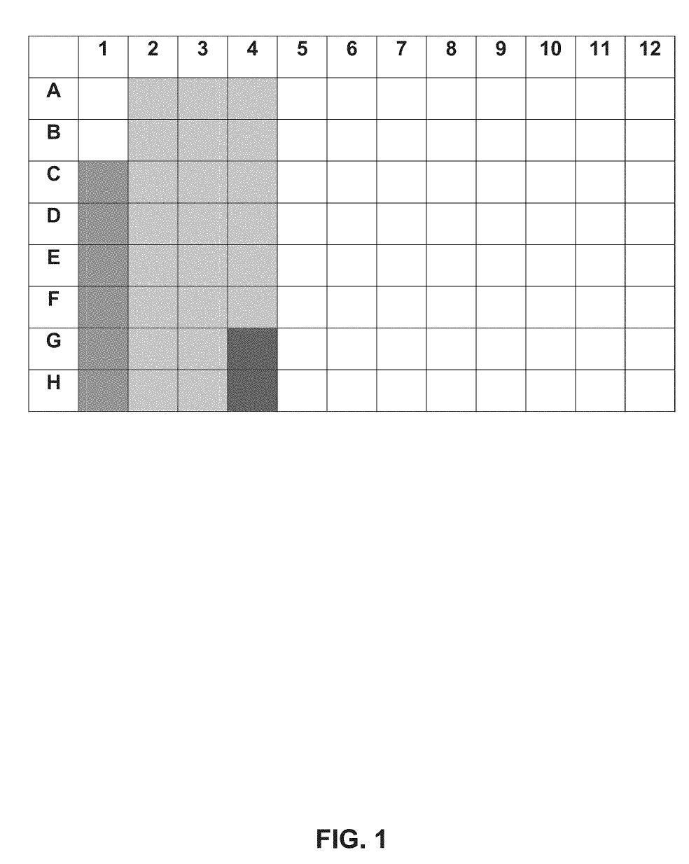

Patient samples (capillary blood or venous blood) containing whole blood first underwent treatment in order to separate serum from whole blood. For example, Microtainers™ containing the patient samples were placed into a centrifuge and then spun at approximately 4° C. for 10 minutes at 5000 rcf. After adequate separation, 150 μL of serum was pipetted from each specimen into one of the wells of a 96-well deep well plate. For serum samples, 150 μL of serum was directly pipetted from each specimen into one of the wells of a 96-well deep well Nunc™ non-silated plate. For each plate containing patient samples, the following other samples were also prepared and placed on the sample plate (see, for example: FIG. 1, discussed hereinbelow): calibrators, QC samples, and blanks (matrix and double blank). The preparation of each of these sample types is discussed hereinbelow.

The starting plate was non-silated. 200 μL of internal standard solution was pipetted into each of the wells containing the patient sample.

The internal standard was made up of isotopically labeled hormones that corresponded to the analyte of interest (Table 1). Deuterium was typically used for the isotopic label in the experiments. However, any non-radioactive isotopic label could potentially be used as an internal standard so long as it possessed a minimum mass difference of M+3 from the analyte of interest.

TABLE 1
Composition of the Internal Standard stock solution.

| Standard | Concentration (ng/ml)* |
|---|---|
| 11-Deoxycortisol-D₅ | 225 |
| 17α-Hydroxyprogesterone-D₈ | 333 |
| 17β-Estradiol-D₅ | 250 |
| Androstene-3,17-dione-2,3,4-¹³C₃ | 225 |
| Cortisol-D₄ | 250 |
| DHEA-D₅ | 625 |
| Progesterone-D₉ | 200 |
| Testosterone-D₃ | 100 |

*In 50:50 v/v LC-MS grade water:methanol.

The plate containing the sample mixture was then placed onto a first mixing device (e.g. a Boekel Autojive™) where the sample was mixed thoroughly. Example conditions for the Boekel Autojive are 1000 rpm for 5 minutes. The plate was then transferred to a laboratory automation system where it underwent supported liquid extraction (SLE). This step involved transferring the patient sample onto an SLE column and performing two sequential elutions with a 60:40 v/v mixture of hexane and methyl t-butyl ether; the first elution contained 900 microliters of the solvent system, and the second elution contained 800 microliters of the same system. These elutions were collected onto a suitable silated 96-well deep well plate It was found that the SLE produced higher recovery with polar organic solvents; however, the use of more non-polar solvents produced a better signal-to-noise ratio on the mass spectrometer due to a cleaner extract which minimized ion suppression and matrix effects even though the recovery was lower than the more polar organic solvents. After completion of the extraction/collection method, the silated 96-well deep well plate containing the patient samples, calibrators and QC materials was dried under nitrogen at approximately 40° C. Samples were then reconstituted in sample diluent (0.5 millimolar NH₄F in 50/50 Methanol/Water). The sample plate was placed onto the mixing device, and the sample and diluent thoroughly mixed. The sample plate was then loaded onto a Shimadzu 8050 LC-MS/MS instrument for analysis.

### (c) Blank Sample Preparation

To ensure adequate quality control practices are followed, two separate types of blanks were used during each sample run,

These blanks were the “double blank” and the “matrix blank”. Each type of blank acted as a measure for potential error or bias. For both types, charcoal stripped fetal bovine serum was used.

The double blank sample was prepared as follows: 150 μL of sample matrix substitute was pipetted into the desired well of the 96-deep well plate, 200 μL of a water/methanol mixture (50/50, v/v) was added to that well, and the sample underwent the same steps described in the “General Sample Preparation Procedure” described in subsection (b) starting with the placement of the 96-well deep well plate onto the first mixing device.

The matrix blank sample was prepared as follows: 150 μL of sample matrix substitute was pipetted into the desired well in the 96-well deep well plate, 200 μL of internal standard solution was then added to that well and the sample underwent the same steps described in the “General Sample Preparation Procedure” described in subsection (b) starting with the placement of the 96-well deep well plate onto the first mixing device.

### (d) Calibrator Sample Preparation

Calibrators for each run were prepared from calibrator solutions (described hereinbelow). Six different levels of calibrators were used. For each individual calibrator, 135 μL of matrix substitute was pipetted into the desired well of the 96-well deep well plate, 200 μL of internal standard solution was then pipetted into that well, 15 μL of the single calibrator was pipetted into the well and the sample underwent the same steps described in the “General Sample Preparation Procedure” described in subsection (b) starting with the placement of the 96-well deep well plate onto the first mixing device.

### (e) Plate Set Up

For all patient samples undergoing analysis, a row of calibrators was used and at least two quality control (QC) samples. The calibrators and QC samples accompanied the patient samples on the plate. FIG. 1 shows a schematic of a sample plate representing one potential configuration. Other variations of this set-up include: variation in number of patient samples; additions in number of blanks, quality control samples, or even additional calibrators, which may be useful in experimental or quality assurance contexts. In FIG. 1, white cells are “blanks”; medium grey colored cells denote calibrators; light grey colored cells denote patient samples; and dark grey colored cells indicate quality control samples. Two levels of external controls were purchased from UTAK (Table 2). These levels contained several hormones (androstenedione, cortisol, cortisone, corticosterone. 11-deoxycortisol, DHEA, progesterone, 17-alpha-hydroxyprogestrone, and testosterone) at known concentrations.

TABLE 2
External Controls.

| | | UTAK |
|---|---|---|
| Hormone | UTAK Level 1 (ng/ml) | Level 3 (ng/ml) |
| Androstenedione | 0.5 | 2.5 |
| Cortisol | 25 | 125 |
| Cortisone | 6 | 30 |
| Corticosterone | 1 | 5 |
| 11-deoxycortisol | 0.4 | 2 |
| DHEA | 1 | 5 |
| Progesterone | 2.5 | 12.5 |
| 17-alpha-hydroxyprogesterone | 1 | 5 |
| Testosterone | 1 | 5 |

Additionally, as discussed in greater detail hereinbelow, calibrators containing all of the analytes of interest were used as internal controls.

### (f) Mobile Phase and Wash Solution Preparation

(i) Mobile Phase A: Water 0.5 mmol Ammonium Fluoride

A clean 2 L volumetric flask or graduated cylinder was filled with LC-MS/MS grade water. 0.037 g (1 millimole) of ammonium fluoride was added, and the mixture stirred until the ammonium fluoride was dissolved.

(ii) Mobile Phase B: Methanol 0.5 mmol Ammonium Fluoride

One 2 liter portion of HPLC-grade methanol was added to a bottle. One millimole (0.037 g) of ammonium fluoride was then weighed out and added to the methanol. The mixture was stirred until the ammonium fluoride dissolved,

(iii) Strong Wash: 60 Isopropanol: 30 Methanol: 10 Water

600 mL LC-MS/MS grade isopropanol, 300 mL LC-MS/MS grade methanol and 100 mL of LC-MS/MS grade water were thoroughly mixed together. The strong wash was used for cleaning the auto-sampler needle between injections.

(iv) Sample Diluent: 0.5 mmol Ammonium Fluoride in 50% LC-MS/MS Grade Methanol, 50% LC/MS-MS Grade Water

500 mL LC-MS/MS grade water was added to a 1 L flask. 0.0185 g (0.5 millimole) of ammonium fluoride was added, and the mixture stirred until dissolved. It was then brought to a 1 L volume with LC-MS Grade Methanol. The mixture was then mixed well by inversion,

### (g) Quality Control Calibrator and Solution

Calibrator Stock Solution was supplied by Sanis Biomedical in Mandeville, La. from certified reference material from Cerilliant™. Chemicals of equivalent purity may be used in place of Cerilliant standards.

The calibrators A-F for a calibration curve were prepared by sequential dilution of “Calibrator Stock” in clean, 12 mL screw-top glass vials with “Calibrator Diluent” (a mixture of 50:50 v/v LC-MS grade water:methanol) to obtain the concentrations (ng/ml) shown in Table 3. The solutions were mixed by closing the vial and inverting several times.

TABLE 3
Concentrations (ng/ml) of hormones within the calibrator stock.

| | A | B | C | D | E | F |
|---|---|---|---|---|---|---|
| | (ng/ml) | (ng/ml) | (ng/ml) | (ng/ml) | (ng/ml) | (ng/ml) |
| 11-Deoxycorticosterone | 50.000 | 25.000 | 12.500 | 6.250 | 3.125 | 1.563 |
| 11-Deoxycortisol | 75.000 | 37.500 | 18.750 | 9.375 | 4.688 | 2.344 |
| 17α- | 100.000 | 50.000 | 25.000 | 12.500 | 6.250 | 3.125 |
| Hydroxyprogesterone | | | | | | |
| 17β-Estradiol | 50.000 | 25.000 | 12.500 | 6.250 | 3.125 | 1.563 |
| 21-Deoxycortisol | 100.000 | 50.000 | 25.000 | 12.500 | 6.250 | 3.125 |
| Aldosterone | 75.000 | 37.500 | 18.750 | 9.375 | 4.688 | 2.344 |
| Androstenedione | 75.000 | 37.500 | 18.750 | 9.375 | 4.688 | 2.344 |
| Corticosterone | 75.000 | 37.500 | 18.750 | 9.375 | 4.688 | 2.344 |
| Cortisol | 100.000 | 50.000 | 25.000 | 12.500 | 6.250 | 3.125 |
| Cortisone | 100.000 | 50.000 | 25.000 | 12.500 | 6.250 | 3.125 |
| DHEA | 400.000 | 200.000 | 100.000 | 50.000 | 25.000 | 12.500 |
| DHT | 50.000 | 25.000 | 12.500 | 6.250 | 3.125 | 1.563 |
| Estriol | 50.000 | 25.000 | 12.500 | 6.250 | 3.125 | 1.563 |
| Estrone | 100.000 | 50.000 | 25.000 | 12.500 | 6.250 | 3.125 |
| Progesterone | 50.000 | 25.000 | 12.500 | 6.250 | 3.125 | 1.563 |
| Testosterone | 50.000 | 25.000 | 12.500 | 6.250 | 3.125 | 1.563 |

* “Calibrator Stock” and all 6 calibrators were stored at −20° C.

When preparing calibrators, 15 microliters of one of the calibrators above was placed into 135 microliters of charcoal stripped fetal bovine serum. This produced a 1:10 dilution. Accordingly, the working concentrations of the analytes in the calibrators were lower than the calibrator stock by a factor of 10.

The calibrators were used to obtain calibration curves. The information obtained from plotting the linear best fit is summarized in Table 4.

TABLE 4
Summary of Calibration Curve Information.

| | R² | Equation of Line | R |
|---|---|---|---|
| 11- | 0.9981606 | 1.08424x + 0 | 0.9990799 |
| deoxycorticosterone | | | |
| 11-Deoxycortisol | 0.9994095 | 0.607191x + 0 | 0.9997047 |
| 17-alpha-OH- | 0.9984951 | 0.0757586x + 0 | 0.9992473 |
| progesterone | | | |
| 17b-estradiol | 0.9965857 | 1.72224X + 0 | 0.9982914 |
| 21-deoxycortisol | 0.9998727 | 0.840509x + 0 | 0.9999363 |
| Aldosterone | 0.9971344 | 0.408348x + 0 | 0.9985662 |
| Androstenedione | 0.999265 | 0.156390x + 0 | 0.9996332 |
| corticosterone | 0.9995173 | 0.399500x + 0 | 0.9997586 |
| Cortisol | 0.9985653 | 2.02465x + 0 | 0.9992824 |
| Cortisone | 0.9980093 | 3.21439x + 0 | 0.9990041 |
| DHEA | 0.9962246 | 0.250496x + 0 | 0.9981105 |
| DHT | 0.9903194 | 0.935439x + 0.0139995 | 0.9951479 |
| Estriol | 0.998399 | 0.767883x + 0 | 0.9991992 |
| Estrone | 0.9972625 | 3.66285x + 0 | 0.9986303 |
| Progesterone | 0.9992182 | 0.523472x + 0 | 0.999609 |
| Testosterone | 0.9980326 | 0.268770x + 0.00575820 | 0.9990158 |

Table 5 summarizes the measurement ranges for each of the analytes in a sample using the methods described herein.

TABLE 5
Measurement Ranges for Hormone Panel.

| | Upper Limit (ng/ml) | Lower Limit (ng/ml) |
|---|---|---|
| 11-deoxycorticosterone | 5 | 0.156 |
| 11-Deoxycortisol | 7.5 | 0.234 |
| 17-alpha-OH-progesterone | 10 | 0.313 |
| 17b-estradiol | 5 | 0.09 |
| 21-deoxycortisol | 10 | 0.313 |
| Aldosterone | 7.5 | 0.234 |
| Androstenedione | 7.5 | 0.234 |
| Corticosterone | 7.5 | 0.234 |
| Cortisol | 250 | 0.313 |
| Cortisone | 60 | 0.313 |
| DHEA | 40 | 1 |
| DHT | 15 | 0.468 |
| Estriol | 5 | 0.156 |
| Estrone | 10 | 0.313 |
| Progesterone | 25 | 0.156 |
| Testosterone | 10 | 0.070 |

### (h) Chromatographic Conditions

Chromatography conditions are summarized in Tables 6 and 7. Two Shimadzu LC-30AD pumps were used for HPLC separation.

TABLE 6
Chromatographic Conditions.

| | |
|---|---|
| LC Stop Time | 9.20 min |
| Mode | Binary Gradient |
| Total Flow Rate | 0.70 mL/min |
| Mobile Phase Start Condition | 60.0% Mobile Phase B |

TABLE 7
HPLC Pump Parameters.

| Time (minutes) | Pump/Mobile Phase B Concentration (% vol) |
|---|---|
| 0.01 | 60 |
| 2.00 | 75 |
| 4.00 | 75 |
| 5.00 | 95 |
| 8.00 | 95 |
| 8.10 | 60 |
| 9.20 | Stop |

### (i) Sample Collection

Conventional systems use venous puncture to collect patient blood samples. Disclosed herein are methods that can measure hormone levels in small volume sample sizes which can be supplied by capillary blood, for example by a fingerprick collection method, which means that a phlebotomist or nurse is not required to obtain the blood sample. This can, for example reduce costs, facilitate compliance and ease in procuring patient samples. It is also demonstrated herein that capillary blood can be used to provide analytical measurements of hormone levels in patient samples (see Example 8).

In contrast to conventional methods, wherein the patient sample needs to be spun and serum isolated prior to transportation for example, for stability and cost effectiveness, the small volumes used in the present method mean that the whole blood sample can be transported.

Further, and also in contrast to conventional systems, where proteins are precipitated prior to analysis, no precipitation of proteins is required in the present method because of the use of SLE, which simplifies the preparation and can improve consistency of the results The SLE columns allow non-polar analytes that contain no readily available/easily ionized groups to elute, while trapping polar components on the column. Given that serum is a complex matrix containing many polar components (water, proteins, phospholipids, electrolytes, etc.) SLE provides a useful purification step for these methods.

### Example 1

### Altering the Voltage Window

Blood samples were obtained and processed as described above and the parameters for LC-MS/MS were as described above unless indicated otherwise.

Table 8 shows voltages and default parameters selected by Shimadzu compared to the settings utilized herein. Expanding the window allowed for identification of optimal voltage assignments for the hormones.

TABLE 8

| | | | | | | |
|---|---|---|---|---|---|---|
| | | | Lower | Upper | Step | |
| Voltage | Optimize | Default | Limit | Limit | Width | Unit |

Standard voltages and default parameters

| | | | | | | | |
|---|---|---|---|---|---|---|---|
| Polarity | Q1 Pre | Yes | Yes | −40.0 | −20.0 | 2.0 | V |
| (+) | Bias | | | | | | |
| | CE | Yes | No | −50.0 | −10.0 | 5.0 | V |
| | Q3 Pre | Yes | Yes | −40.0 | −20.0 | 2.0 | V |
| | Bias | | | | | | |
| Polarity | Q1 Pre | Yes | Yes | 20.0 | 40.0 | 2.0 | V |
| (−) | Bias | | | | | | |
| | CE | Yes | No | 10.0 | 50.0 | 5.0 | V |
| | Q3 Pre | Yes | Yes | 20.0 | 40.0 | 2.0 | V |
| | Bias | | | | | | |
| Optimize | CE | Yes | No | −5.0 | 5.0 | 1.0 | V |
| results | | | | | | | |
| with the | | | | | | | |
| details | | | | | | | |

Voltages and default parameters used herein

| | | | | | | | |
|---|---|---|---|---|---|---|---|
| Polarity | Q1 Pre | Yes | No | −50.0 | −10.0 | 2.0 | V |
| (+) | Bias | | | | | | |
| | CE | Yes | No | −50.0 | −10.0 | 5.0 | V |
| | Q3 Pre | Yes | No | −50.0 | −10.0 | 2.0 | V |
| | Bias | | | | | | |
| Polarity | Q1 Pre | Yes | No | 10.0 | 50.0 | 2.0 | V |
| (−) | Bias | | | | | | |
| | CE | Yes | No | 10.0 | 50.0 | 5.0 | V |
| | Q3 Pre | Yes | No | 10.0 | 50.0 | 2.0 | V |
| | Bias | | | | | | |
| Optimize | CE | Yes | No | −5.0 | 5.0 | 1.0 | V |
| results | | | | | | | |
| with the | | | | | | | |
| details | | | | | | | |

### Example 2

### Effects of Interface Voltage

### (a) Materials and Methods

The effect of different voltages than those stored in the default instrumentation tuning parameters on sensitivity was investigated.

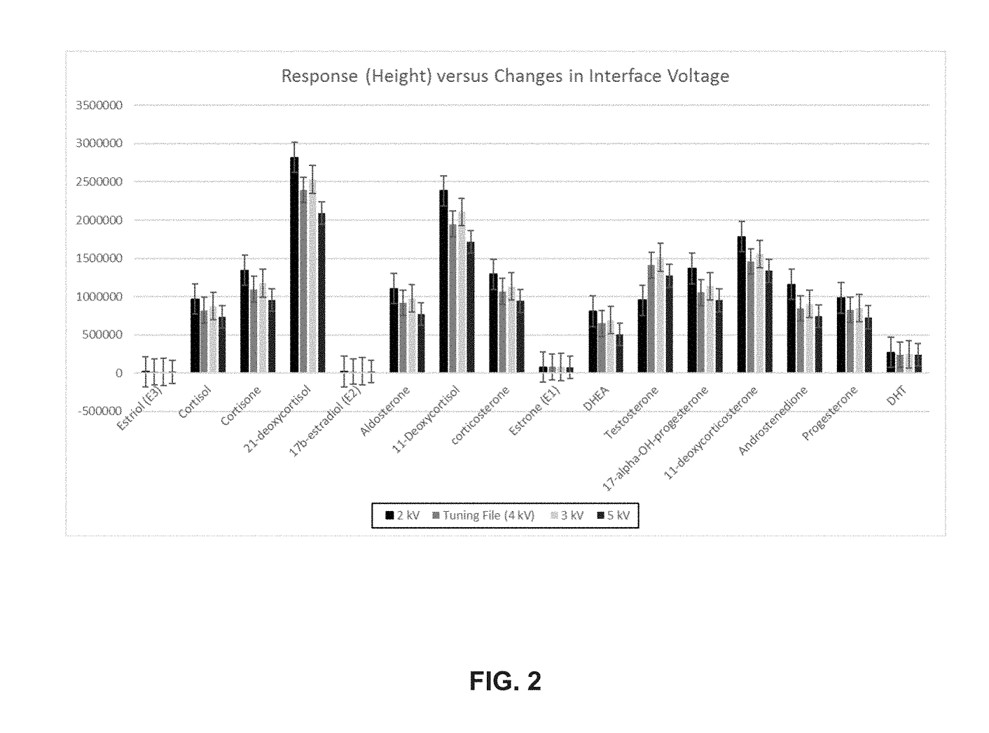

FIG. 2 depicts an experiment showing the increase in response due to changes in interface voltage (i.e. taking the machine out of tune). All other parameters were kept the same. A 1:10 dilution of the second highest calibrator was selected to be measured, and an injection volume of 20 microliters was used. Two experimental runs were completed per each voltage chosen.

### (b) Results and Discussion

LC-MS/MS typically utilize the tune or mass calibration file parameters to achieve maximum sensitivity. By default, the instrument utilizes the tuning parameters to meet adequate sensitivity and resolution.

FIG. 2 shows the average height of analytes at different interface voltages. Each voltage was tested the same number of times; and the average of those runs was plotted, along with the standard deviation (error bar).

The present method examined the effect of turning the voltage down and up from the tuning file average of 4 kV during measurement. One would expect turning the voltage down to result in lower response signals. However, FIG. 2 demonstrates the lower voltage produced better results for many analytes. For each analyte, the voltage change to a lower value (2 or 3 kV) produced anywhere from a 5-15% increase in signal intensity (height). Table 9 shows the average response for each of the interface voltages tested.

TABLE 9
The Average Response (Height) at Interface Voltages Tested.

| | 2 kV | 3 kV | 4 kV (Tune) | 5 kV |
|---|---|---|---|---|
| Estriol (E3) | 19099 | 18498 | 18638 | 17207.5 |
| Cortisol | 967299 | 875683 | 821925 | 738325 |
| Cortisone | 1342586 | 1175587 | 1097455 | 958852.5 |
| 21-deoxycortisol | 2818257 | 2531564 | 2391672 | 2091502 |
| 17b-estradiol (E2) | 21358 | 22986.5 | 21922.5 | 22229 |
| Aldosterone | 1103583 | 976630 | 918606 | 775896 |
| 11-Deoxycortisol | 2382630 | 2108002 | 1948944 | 1719124 |
| corticosterone | 1290673 | 1131019 | 1068162 | 942666.5 |
| Estrone (E1) | 79852 | 82757 | 82003 | 78029.5 |
| DHEA | 808294 | 691656.5 | 649032.5 | 508229.5 |
| Testosterone | 952624 | 1514384 | 1410240 | 1272616 |
| 17-alpha-OH- | 1366916 | 1135706 | 1053889 | 951893 |
| progesterone | | | | |
| 11- | 1784585 | 1557418 | 1460183 | 1336146 |
| deoxycorticosterone | | | | |
| Androstenedione | 1159436 | 902658 | 847918 | 743117.5 |
| Progesterone | 983480 | 854477.5 | 825961.5 | 730701 |
| DHT | 271131 | 249137.5 | 242356.5 | 243346.5 |

### Example 3

### Effect of Desolvation Line (DL) Temperature Variation

### (a) Materials and Methods

Blood samples were obtained and processed as described above unless indicated otherwise. The parameters for LC-MS/MS were as described above unless indicated otherwise.

DL temperatures of 140° C., 250° C. and 300° C. were compared to the normal operating temperature of 200° C. All other parameters were set to instrument standards for this study. A 1:10 dilution of the second highest calibrator was selected to be measured, and an injection volume of 20 microliters was used. Two experimental runs were completed per each voltage chosen.

### (b) Results and Discussion

Suggested desolvation line (DL) temperatures of 200° C. or greater often improve the signal-to-noise ratio (S/N) by reducing solvent noise and improving ion transfer into the quadrupoles and optics.

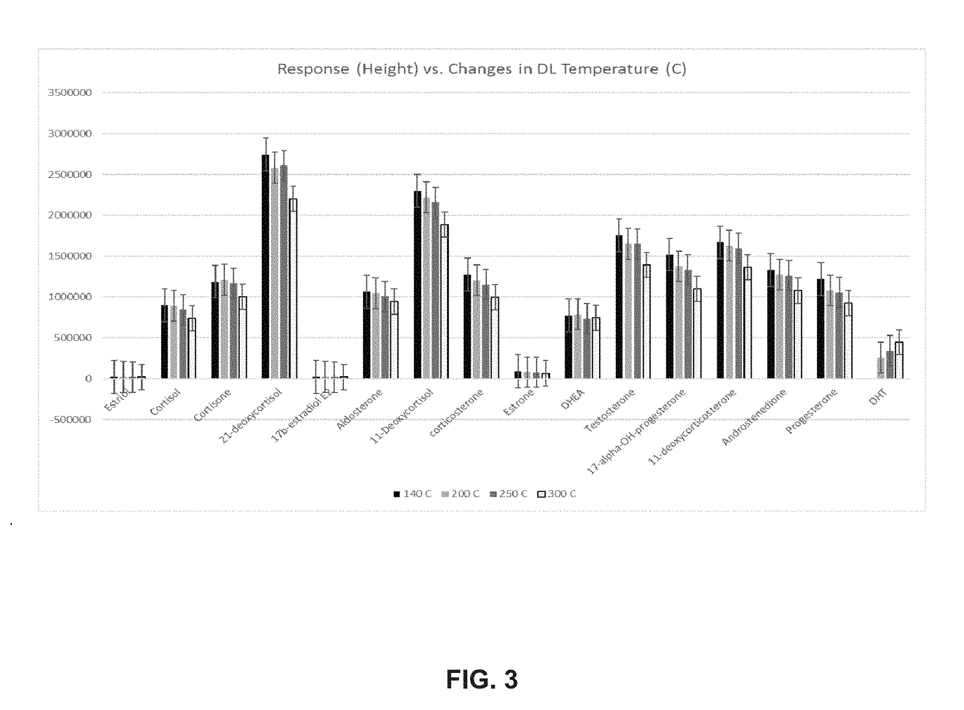

Contrary to recommendation (or normal operation), it was found that decreases in DL temperature generally led to better results for the hormones being analyzed. For example, FIG. 3 demonstrates how increasing DL temperature generally reduces signal and ion count on the detector. The average height response of analytes are plotted against their respective DL temperature. For some of these analytes, e.g. DHT, the response increases with temperature and for others, e.g. DHEA, the response increases then decreases again. However, the total results suggest that 140° C. is a useful temperature to use for a method for detecting all of these analytes (e.g. in a multiplex; looking at all of these hormones within a single run) because even though signal response is lower for some, in general across the board, it is a better temperature for most of them.

### Example 4

### Unique Transitions

### (a) Background

Positive ionizing MRMs utilize the M+H, where M is equal to the mass of the analyte to be quantitated by the mass spectrometer. In the event a compound ionizes in the negative mode, the M−H is utilized as the m/z value for the analyte. Furthermore, the software default parameters as well as common practice is to select the 2 MRMs with the highest signal.

The present example described in greater detail below, follows an unconventional approach for setting up the MRM of two compounds.

### (b) Dehydroepiandrosterone (DHEA)

Samples were obtained and processed as described above. The parameters for LC-MS/MS were as described above unless indicated otherwise.

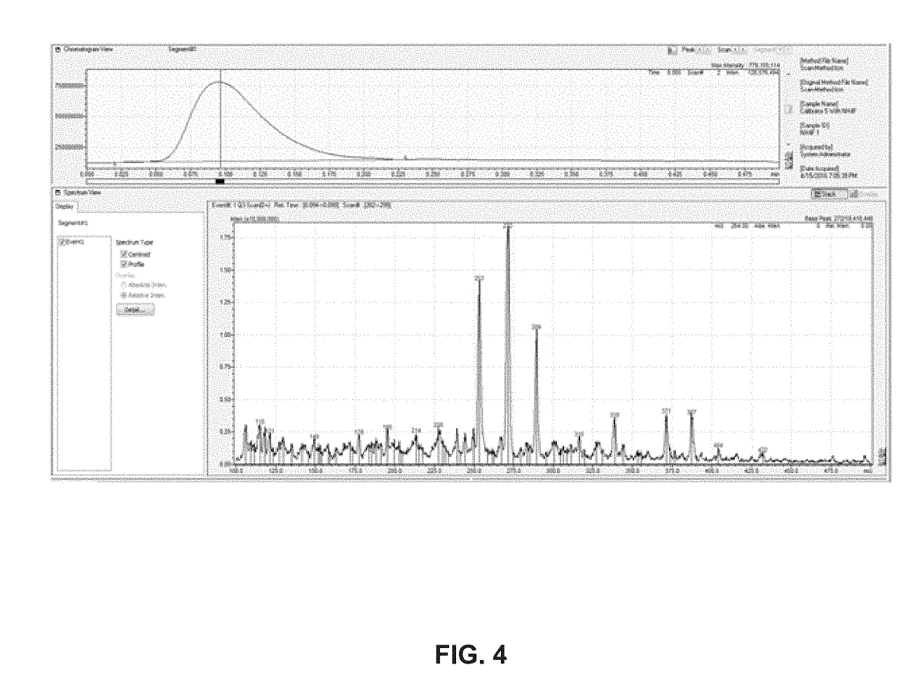

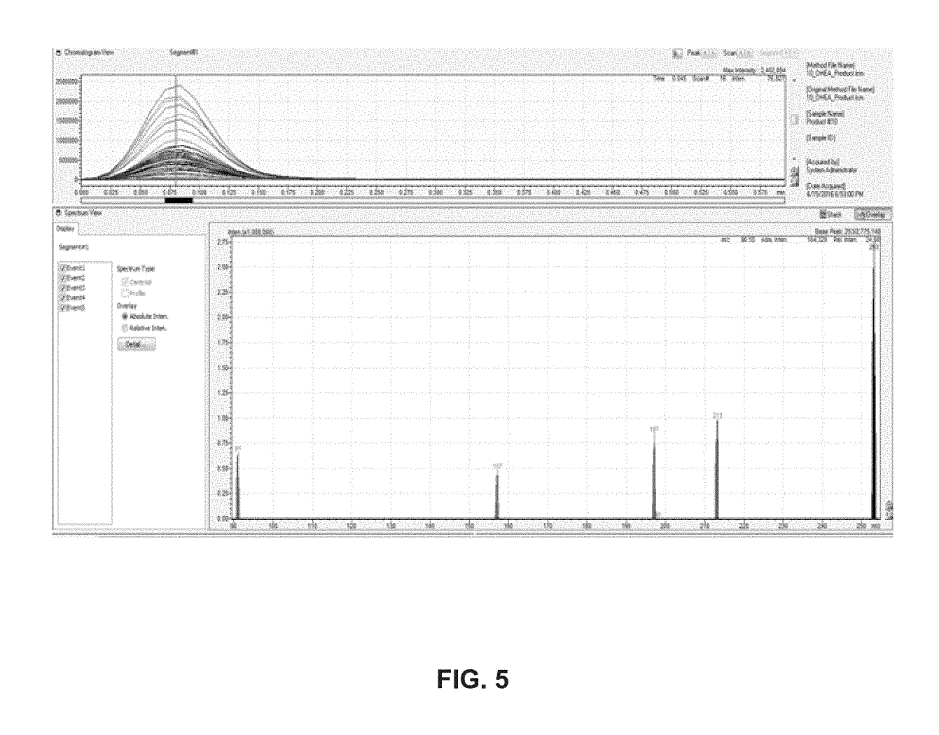

DHEA appeared to be unstable to electrospray ionization (ESI). A precursor ion scan (FIG. 4) demonstrated three different ions within a certified reference material (CRM) from Cerillant. The M+H (m/z=289) was the least abundant ion, followed by the commonly used transition of (m/z=253) (Kushnir et al. 2010; Lewis et al. 2013). The (M+H)—(H₂O) ion (m/z=271) was accordingly used for the precursor ion. The most abundant transition was the 271→253 transition, but this transition was not used. Instead, the lower mass transition of 271→213 was used, which worked counter-intuitive to a normal process. Using this transition resulted in less noise and better sensitivity. FIG. 5 shows the chromatogram (top panel) and MRM (bottom panel) showing the most common ions associated with the abovementioned transitions. The combination of the above two techniques created a more sensitive transition that maintained specificity.

### (c) Aldosterone

Samples were obtained and processed as described above. The parameters for LC-MS/MS were as described above unless indicated otherwise.

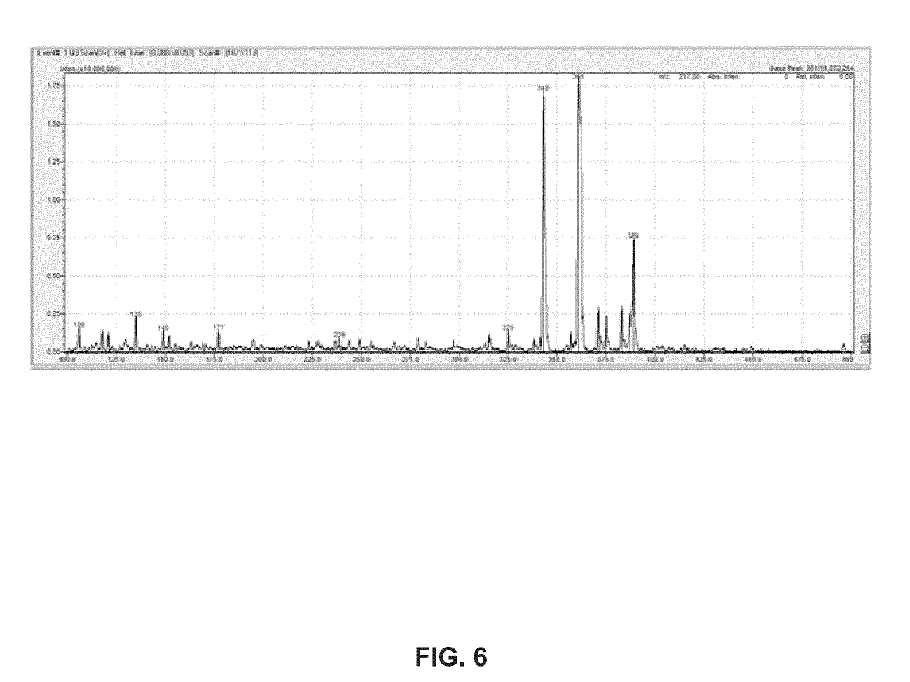

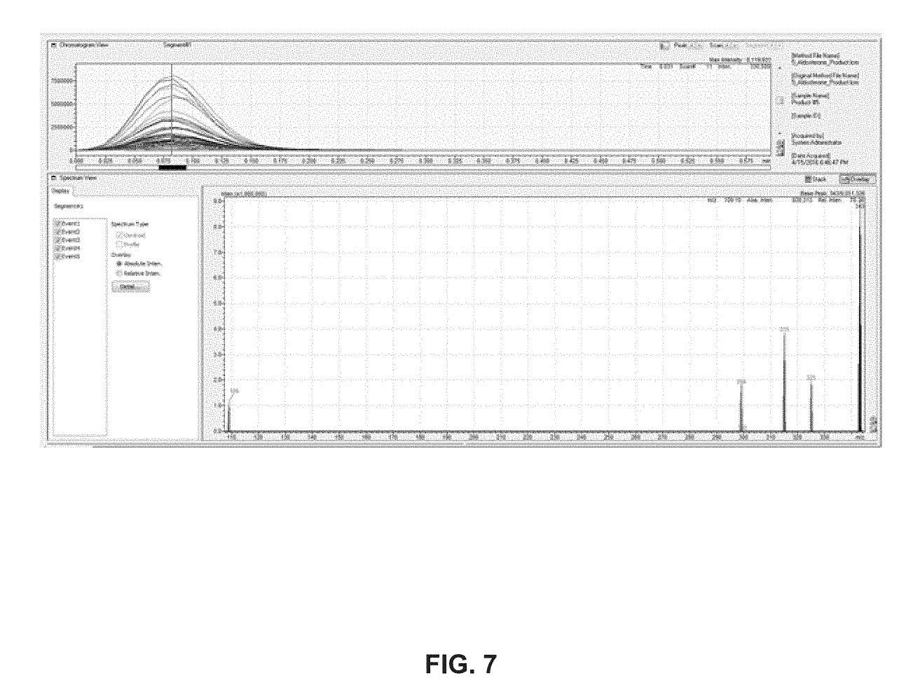

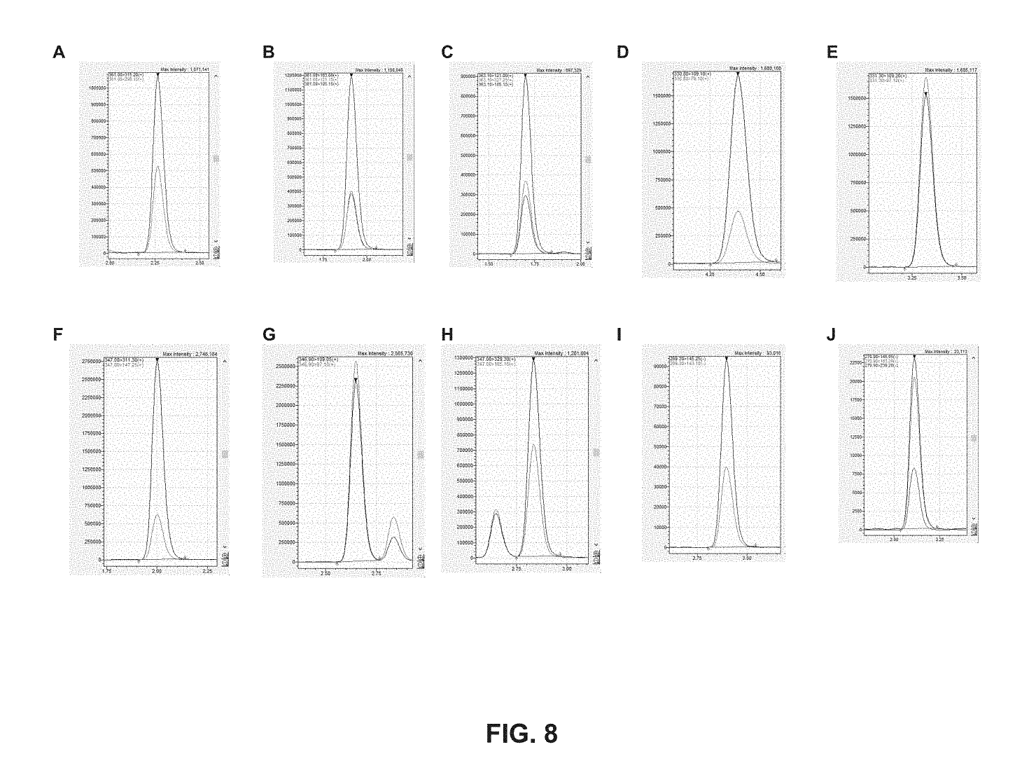

For this compound, the M+H precursor ion (m/z=361) was used (FIG. 6). However, the two most abundant product ions were not used because they are water loss transitions and instead, a product ion of m/z=315.2 was used. Furthermore, aldosterone is typically analyzed in negative mode (Taylor et al. 2009; Turpeinen et al. 2008), but positive mode was used for the MRM and achieved useful sensitivity. FIG. 6 shows an exemplary chromatogram (top panel) and MRM (bottom panel) which was obtained for aldosterone.

### Example 5

### Effect of Using NH₄F as a Sample Diluent and Mobile Phase

### (a) Materials and Methods

Samples were obtained and processed as described above. The interface settings used are set out in Table 10.

TABLE 10
Interface Settings.

| | |
|---|---|
| Interface | DUIS-ESI |
| Interface Heater | On |
| Interface Temperature | 400° C. |
| DL Temperature | 140° C. |
| Nebulizing Gas Flow | 3.00 L/min |
| Heating Gas | On |
| Heating Gas Flow | 11.00 L/min |
| Heat Block Temperature | 500° C. |
| Drying Gas | On |
| Drying Gas Flow | 9.00 L/min |

### (b) Results and Discussion

Hormones are non-polar molecules with few easily ionizable functional groups. In many applications, weak acids (e.g. formic acid) have been used. NH₄F is an uncommon additive to a mobile phase. The few reported cases consist of utilizing it in a single mobile phase only. Conventional methods may also use a 50:50 mixture of mobile phase A (MPA) and mobile phase B (MPB) without NH₄F.

A modest increase on some hormones was observed by utilizing NH₄F in the sample diluent as well as both mobile phases, including for example some hormones typically found in low concentration. The enhancement in the signal to noise ratio was significant (Table 11).

TABLE 11
Effect of the presence of NH₄F on the signal to noise ratio for
various hormones and standards.

| | Ratio | Difference |
|---|---|---|
| Aldosterone | 0.71 | 29.50 |
| Cortisol-D₄ | 0.85 | 15.11 |
| Cortisone | 0.88 | 12.31 |
| Cortisol | 0.89 | 10.78 |
| Estriol | 0.95 | 4.85 |
| 17b-Estradiol | 1.00 | −0.35 |
| 17-alpha-OH-Progesterone | 1.03 | −2.71 |
| Corticosterone | 1.05 | −4.63 |
| 17-alpha-OH-Progesterone-D₈ | 1.05 | −4.97 |
| Testosterone | 1.06 | −5.82 |
| 17b-Estradiol-D₅ | 1.09 | −9.40 |
| 11-Deoxycortisol-D₅ | 1.10 | −10.46 |
| Testosterone-D3 | 1.11 | −10.68 |
| 21-Deoxycortisol | 1.11 | −10.94 |
| 11-Deoxycortisol | 1.12 | −11.96 |
| Estrone | 1.14 | −13.84 |
| DHT | 1.21 | −21.16 |
| DHEA | 1.27 | −27.27 |
| 11-Deoxycorticosterone | 1.29 | −28.59 |
| DHT D₃ | 1.30 | −29.76 |
| Androstene-3,17-dione ¹³C₃ | 1.37 | −36.80 |
| DHEA-D₅ | 1.37 | −37.31 |
| Progesterone-D₉ | 1.39 | −38.61 |
| Progesterone | 1.39 | −38.72 |
| Androstenedione | 1.49 | −48.86 |

These results suggest that using NH₄F would be useful for a method for detecting all of these analytes (multiplexing) because in general across the board, it provides better conditions for most of them.

### Example 6

### Chromatography

### (a) Materials and Methods

Samples were obtained and processed as described above. Chromatographic conditions were as detailed hereinabove in the general materials and methods section of the Examples.

### (b) Results and Discussion

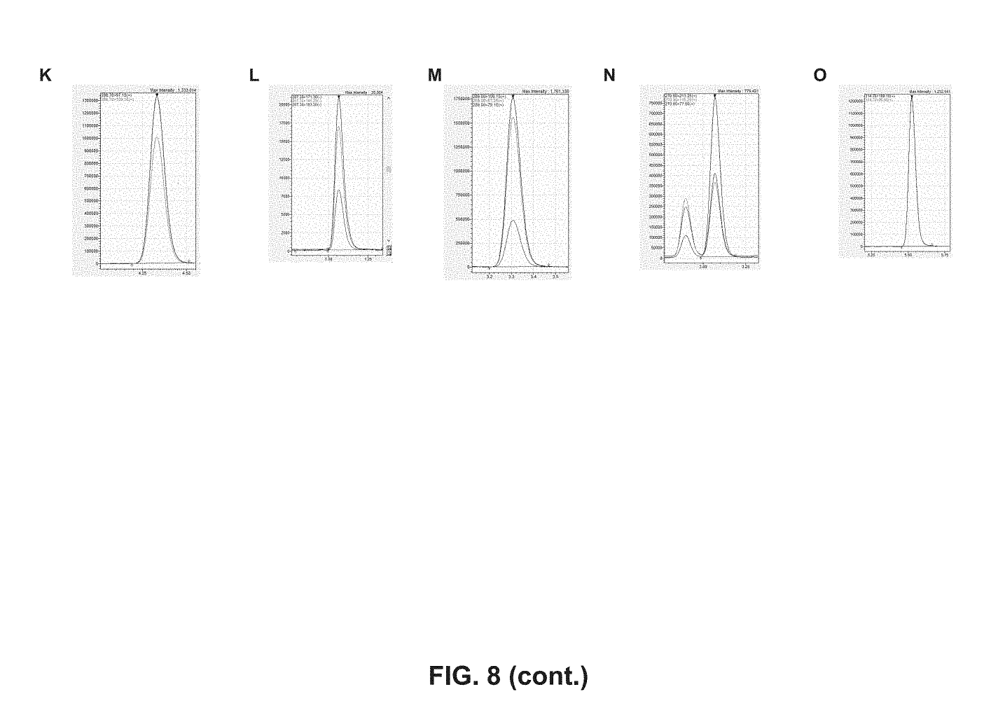

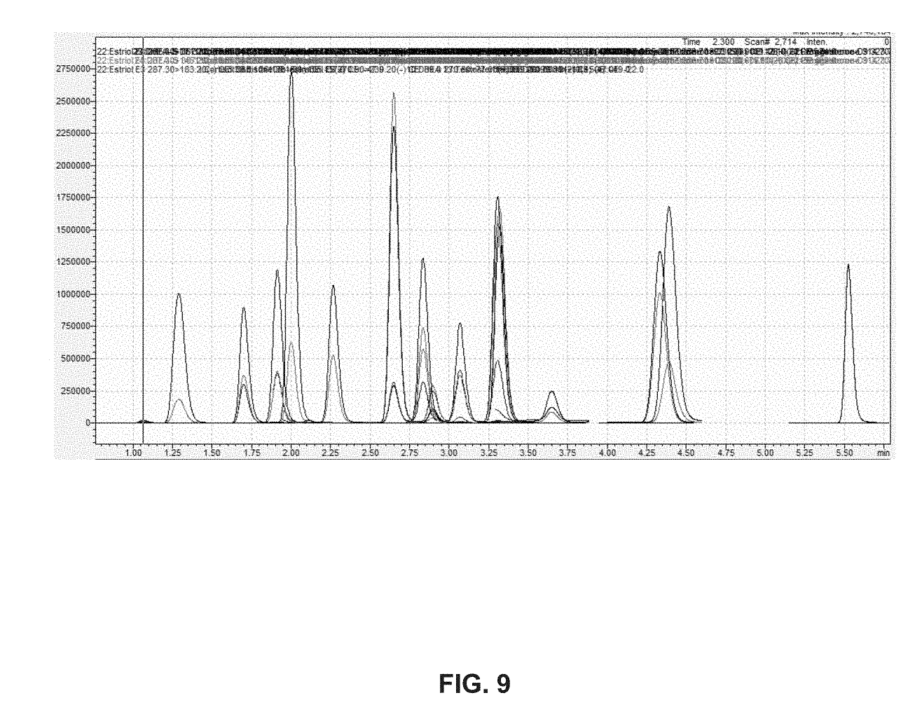

One of the challenges in multiplexing analytes is dealing with isobars (molecules or ions with the same atomic mass). Given that many hormones are structurally similar and isobaric, chromatographic resolution can play a useful role in the methods. Tables 12 and 13 give details as to molecular mass and retention times for each of the analytes studied. Chromatograms obtained under these conditions showed that isobaric resolution occurs (FIGS. 8-9). As one can see from the tables below, many of these hormones are isobaric, and are known to have very similar structures. The fact that they could be chromatographically distinguished was striking.

TABLE 12
Mass and Retention Time for Corticosteroids.

| Hormone | Mass (amu) | Retention Time (minutes) |
|---|---|---|
| aldosterone | 360.44 | 2.26 |
| cortisone | 360.44 | 1.919 |
| cortisol | 362.46 | 1.713 |
| 11-deoxycorticosterone | 330.46 | 4.294 |
| 17-α-hydroxyprogesterone | 330.46 | 3.293 |
| 21-deoxycortisol | 346.46 | 2.004 |
| 11-deoxycortisol | 346.46 | 2.629 |
| corticosterone | 346.47 | 2.812 |

As can be seen from the above table, useful differences in retention time during the high performance liquid chromatography portion of the method were observed for corticosteroids having similar molecular masses. Different retention times allow for the mass spectrometer to more specifically identify analytes that would otherwise be difficult for the machine to distinguish.

TABLE 13
Mass and Retention Time for Sex Steroids and Progesterone

| Hormone | Mass (amu) | Retention Time (minutes) |
|---|---|---|
| Estrone | 270.36 | 2.868 |
| Estradiol | 272.38 | 2.112 |
| Androstenedione | 286.40 | 4.245 |
| Estriol | 288.38 | 1.066 |
| Testosterone | 288.42 | 3.286 |
| DHEA | 288.42 | 3.042 |
| DHT | 290.44 | 3.612 |
| Progesterone | 314.46 | 5.477 |

As can be seen from the above table, useful differences in retention time during the chromatography (HPLC) portion of the method were observed for sex steroids having similar molecular masses and progesterone.

FIG. 8 shows chromatograms for each of the individual hormones. FIG. 9 shows a chromatogram of the entire run. The total time for the run was 9.2 minutes. The chromatogram shows good separation that was even more remarkable when examined closer. Known methods often use a lower flow rate to obtain better separation. In contrast, the present method uses a higher flow rate and achieves better specificity through sharper peaks.

### Example 7

### Specificity & Resolution Variability

### (a) Materials and Methods

Samples were obtained and processed as described above. Chromatographic conditions were as detailed hereinabove in the general materials and methods section of the Examples. DL Temperature was140° C.

### (b) Results and Discussion

High performance liquid Chromatography-tandem mass spectrometry (LC-MS/MS) utilizing multiple reaction monitoring (MRM) incorporates the use of mass transitions to identify analytes of interest.

Voltages of the first mass filter (Q1), collision energy (CE) and second mass filter (Q3) are determined from the automated MRM software on an LC-MS/MS system. Typically, the Interface and dual ion source (DUIS) voltages are pre-selected from the tune file. Triple quadrupole mass-spectrometers typically utilize unit resolution for their m/z identification.

Example 1 describes how the voltages that were scanned by the software platform were changed. Due to those specific changes, the optimal voltages for Q3 or product ions were able to be identified which made the sensitivity of the present technique possible. Furthermore, the interface voltage was taken out of tune and brought to the lowest possible setting (2 kV).

The Q1/Q3 resolution which is typically set at “unit” was changed according to the individual analyte to improve sensitivity. The variations allowed the instrument to span the analytical range for each method.

The Shimadzu 8050 can to switch from positive mode to negative mode in milliseconds. Positive mode (or positive ion mode) uses negative voltages to focus positive ions. Negative mode (negative ion mode) conversely uses positive voltages in order to focus negative ions towards the detector. Some instruments may require the sample to be analyzed twice—once in positive mode, and another time in negative ion mode. The polarity switching capability of the Shimadzu 8050 was used for measuring estradiol and testosterone.

Table 14 provides a summary of parameters tested.

TABLE 14
Summary of Parameters for Each of the Hormones

| | | | | | | | | |
|---|---|---|---|---|---|---|---|---|
| | | | | | | | Interface | Duis |
| Precursor | Product | Q1 Pre | | Q3 Pre | Q1 | Q3 | Volt. | Volt. |
| m/z | m/z | Bias (V) | CE (V) | Bias (V) | Res. | Res. | (kV) | (kV) |

Aldosterone

| | | | | | | | | | |
|---|---|---|---|---|---|---|---|---|---|
| Ch 1 | 361 | 315.2 | −30 | −20 | −22 | Low | Low | 2 | Tune |
| Ch 2 | 361 | 299.15 | −30 | −25 | −21 | | | | |

Androstenedione

| | | | | | | | | | |
|---|---|---|---|---|---|---|---|---|---|
| Ch 1 | 286.7 | 97.15 | −36 | −26 | −18 | Unit | Unit | 2 | 2 |
| Ch 2 | 286.7 | 109.1 | −36 | −26 | −20 | | | | |

Corticosterone

| | | | | | | | | | |
|---|---|---|---|---|---|---|---|---|---|
| Ch 1 | 347 | 329.3 | −14 | −16 | −22 | Low | Unit | 2 | 2 |
| Ch 2 | 347 | 105.15 | −14 | −45 | −42 | | | | |

Cortisol

| | | | | | | | | | |
|---|---|---|---|---|---|---|---|---|---|
| Ch 1 | 363.1 | 121 | −26 | −28 | −20 | Unit | Unit | 2 | 2 |
| Ch 2 | 363.1 | 327.25 | −26 | −17 | −48 | | | | |
| Ch 3 | 363.1 | 105.15 | −26 | −37 | −42 | | | | |

Cortisone

| | | | | | | | | | |
|---|---|---|---|---|---|---|---|---|---|
| Ch 1 | 361 | 163 | −28 | −25 | −16 | Unit | Unit | 2 | 2 |
| Ch 2 | 361 | 121.15 | −28 | −33 | −12 | | | | |
| Ch 3 | 361 | 105.15 | −28 | −47 | −44 | | | | |

21-Deoxycortisol

| | | | | | | | | | |
|---|---|---|---|---|---|---|---|---|---|
| Ch 1 | 347 | 311.3 | −26 | −18 | −22 | Low | Unit | 2 | 2 |
| Ch 2 | 347 | 147.25 | −26 | −30 | −10 | | | | |

11-Deoxycortisol

| | | | | | | | | | |
|---|---|---|---|---|---|---|---|---|---|
| Ch 1 | 346.9 | 109.05 | −26 | −31 | −18 | Low | Unit | 2 | 2 |
| Ch 2 | 346.9 | 97.1 | −26 | −30 | −38 | | | | |

DHEA

| | | | | | | | | | |
|---|---|---|---|---|---|---|---|---|---|
| Ch 1 | 270 8 | 213.25 | −40 | −17 | −22 | Low | Low | 2 | Tune |
| Ch 2 | 270.8 | 115.2 | −40 | −80 | −46 | | | | |
| Ch 3 | 270.8 | 77 | −40 | −70 | −26 | | | | |

11-Deoxycorticosterone

| | | | | | | | | | |
|---|---|---|---|---|---|---|---|---|---|
| Ch 1 | 330.8 | 109.1 | −26 | −25 | −20 | Low | Unit | 2 | 2 |
| Ch 2 | 330.8 | 79.1 | −26 | −52 | −30 | | | | |

Estrone*

| | | | | | | | | | |
|---|---|---|---|---|---|---|---|---|---|
| Ch 1 | 269.3 | 145.25 | 30 | 40 | 28 | Low | Unit | −2 | Tune |
| Ch 2 | 269.3 | 143.1 | 30 | 55 | 50 | | | | |

17-β-Estradiol*

| | | | | | | | | | |
|---|---|---|---|---|---|---|---|---|---|
| Ch 1 | 270.9 | 145.05 | 20 | 40 | 26.8 | Low | Low | −2 | Tune |
| Ch 2 | 270.9 | 183.2 | 20 | 42 | 50 | | | | |
| Ch 3 | 270.9 | 239.2 | 20 | 39 | 25.5 | | | | |

Estriol*

| | | | | | | | | | |
|---|---|---|---|---|---|---|---|---|---|
| Ch 1 | 287.3 | 171.3 | 14 | 34 | 36 | Low | Low | −2 | Tune |
| Ch 2 | 287.3 | 145.25 | 14 | 39 | 50 | | | | |
| Ch 3 | 287.3 | 183.3 | 14 | 38 | 12 | | | | |

17-α-Hydroxyprogesterone

| | | | | | | | | | |
|---|---|---|---|---|---|---|---|---|---|
| Ch 1 | 331.3 | 109.2 | −26 | −29 | −20 | High | High | 2 | 2 |
| Ch 2 | 331.3 | 97.1 | −26 | −24 | −38 | | | | |

Progesterone

| | | | | | | | | | |
|---|---|---|---|---|---|---|---|---|---|
| Ch 1 | 314.7 | 109.1 | −38 | −27 | −46 | Unit | Unit | 2 | 2 |
| Ch 2 | 314.7 | 96.95 | −38 | −24 | −18 | | | | |

Testosterone

| | | | | | | | | | |
|---|---|---|---|---|---|---|---|---|---|
| Ch 1 | 289 | 109.15 | −22 | −24 | −20 | Unit | Unit | 2 | 2 |
| Ch 2 | 289 | 97.25 | −22 | −30 | −38 | | | | |
| Ch 3 | 289 | 79.1 | −22 | −47 | −32 | | | | |

*Indicates negative ion mode; other hormones run in positive ion mode

The instrument tracked all of the transitions for each analyte (each transition is labeled “Ch” as channel in Table 14).

### Example 8

### Comparison Between Venous and Capillary Blood Samples

### (a) Materials and Methods

Samples were collected from volunteers (n=9, average age=30.5+/−4.96 years). Five samples were taken from females, and four from males. Multiple analyses were run on the samples, resulting in the data points which follow. Care was taken to ensure that both venous puncture and capillary collection took place within approximately 15 minutes of each other. Both capillary and venous collections were put into K2EDTA coated containers. Capillary and venous blood samples were processed as described hereinabove.

### (b) Results and Discussion

Table 15 provides a summary of the degree of correlation as described by the Pearson coefficient between the venous and capillary samples.

TABLE 15
Correlation (Pearson's r) of Venous Draw vs. Capillary Puncture

| Hormone | Pearson's r |
|---|---|
| Cortisol | 0.973 |
| Cortisone | 0.973 |
| 11-deoxycortisol | 0.986 |
| Corticosterone | 0.975 |
| 11-deoxycorticosterone | 0.785 |
| Androstenedione | 0.94 |
| DHEA | 0.83 |
| Testosterone | 0.987 |
| Estrone | 0.923 |
| Estradiol | 0.844 |
| 17-alpha-hydroxyprogesterone | 0.896 |
| Progesterone | 1 |

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR REFERENCES REFERRED TO IN THE SPECIFICATION

Barwick, V.; Langley, J.; Mallet, T.; Stein, B.; Webb, K. Best Practice for Generating Mass Spectra. LGC (Teddington) Limited: Great Britain, 2006
De Hoffmann, E. Tandem Mass Spectrometry: A Primer. Journal of Mass Spectrometry. 1995, 31, 129-137.
Grebe, S K G; Singh, R J. LC-MS/MS in the Clinical Laboratory—Where to From Here? 2011 Clin Biochem Rev 32, 5-31.
Ho, C S; Lam, C W K; Chan, M H M; Cheung, R C K; Lawdolan, L K; Lit, L C W; Ng, K F; Suen, M W M; Tai, H L. Electronspray Ionisation Mass Spectrometry: Principles and Clinical Applications. Clin Biochem Rev. 2003, 24, 3-12.
Nguyen, H. Estrogen Analysis by Liquid Chromatography—Mass Spectrometry. Ph.D. Dissertation, University of Texas at Arlington, Tex., December 2010.
Gervasomi, J.; Schiattarella, A.; Hornshaw, M. Development of a TurboFlow LC-MS/MS Method for Quantitation of 17-hydroxyprogesterone in Human Serum. ThermoScientific, 2014 (Accessed on Apr. 16, 2016).
Kushnir, M. M.; Blamires, T.; Rockwood, A. L.; Roberts, W. L.; Yue, B.; Erdogan, E.; Bunker, A. M.; Meikle, A. W. Liquid Chromatography-Tandem Mass Spectrometry Assay for Androstenedione, Dehydroepiandrostenedione, and Testosterone with Pediatric and Adult Reference Intervals. Clin. Chemistry. 2010, 56, 1138-1147.
Lewis, K. Advances in Steroid Panel Analysis with High Sensitivity LC/MS/MS. Poster presented at MSACL; 2013 Feb. 9-13; San Diego, Calif. USA.
Sawant, D.; Damale, S.; Bhandarkar, D.; Rane, S.; Kochhar, R.; Raju, S.; DAtar, A.; Rasam, P.; Kelkar, J.; Chopra, S.; Clifford, R H. Analysis of Steroids in Milk using QuEChERS Sample Preparation with LC-MS/MS. Shimadzu Journal. http://www.ssi.shimadzu.com/products/literature/lcms/POSTER_AOAC_Steroids_milk_Ver3-8.pdf (Accessed on Apr. 16, 2016).
Taylor, P. J.; Cooper, D. P.; Gordon, R. D.; Stowasser, M. Measurement of Aldosterone in Human Plasma by Semiautomated HPLC-Tandem Mass Spectrometry. Clin. Chemistry. 2009, 55(6), 1155-1162.
Turpeinen, U., Hämalainen, E.; Stenman, U. H. Determination of Aldosterone in Serum by Liquid Chromatography-tandem mass spectrometry. Journal of Chromatography B. 2008, 862, 113-118.

1. A method for determining the concentration of one or more analytes in a sample by liquid chromatography-tandem mass spectrometry (LC-MS/MS), the method comprising:
subjecting at least a portion of the sample to LC-MS/MS under conditions to generate a precursor ion for each of the one or more analytes and to generate one or more product ions from each of the precursor ions;
detecting the amount of the one or more product ions; and
relating the amount of the one or more detected product ions to the concentration of the one or more analytes in the sample,
wherein
the conditions for the liquid chromatography comprise a binary gradient using a first mobile phase and a second mobile phase, both mobile phases comprising ammonium fluoride:
the conditions for the tandem mass spectrometry comprise a desolvation line temperature of less than 200° C.; and/or
the conditions for the tandem mass spectrometry comprise an interface voltage of less than about 4 kV.

2. The method of claim 1, wherein the sample is a biological sample, optionally wherein the sample is a urine sample or a blood sample optionally selected from a whole blood sample, a serum sample and a plasma sample.

3. The method of claim 1 or 2, wherein the sample is a capillary blood sample.

4. The method of claim 2 or 3, wherein the sample is a serum sample and, prior o being subjected to LC-MS/MS, is prepared by a method comprising:
loading the sample onto a supported liquid extraction (SLE) column and eluting with an elution solvent under conditions to collect the one or more analytes in the elution solvent on a sample plate;
drying under conditions to remove the elution solvent; and
reconstituting the sample in a sample diluent comprising ammonium fluoride.

5. The method of claim 4, wherein about 150 μL of the serum sample is loaded onto the SLE column.

6. The method of claim 4 or 5, wherein the sample diluent consists essentially of about 0.25 mM to about 2 mM NH₄F in a methanol-water solution.

7. The method of claim 6, wherein the sample diluent consists essentially of about 0.5 mm NH₄F in in about 50:50 v/v methanol:water.

8. The method of any one of claims 4 to 7, wherein the sample plate is a silated 96-well deep well plate.

9. The method of claim 2 or 3, wherein the sample is a whole blood sample and the method further comprises preparing a serum sample from the whole blood sample by a method comprising centrifuging the whole blood sample under conditions to separate the serum from blood cells, optionally by removing clotted blood cells.

10. The method of any one of claims 1 to 9, wherein the first mobile phase consists essentially of an aqueous solution of from about 0.25 mM to about 2 mM, optionally about 0.5 mM ammonium fluoride.

11. The method of any one of claims 1 to 10, wherein the second mobile phase consists essentially of a methanolic solution of from about 0.25 mM to about 2 mM, optionally about 0.5 mM ammonium fluoride.

12. The method of any one of claims 1 to 11, wherein the conditions for the high performance liquid chromatography comprise a total flow rate of from about 0.50 mL/minute to about 1.0 mL/minute or about 0.70 mL/minute.

13. The method of any one of claims 1 to 12, wherein the one or more analytes are steroid hormones.

14. The method of claim 13, wherein the one or more analytes are corticosteroids, sex steroids or combinations thereof.

15. The method of any one of claims 1 to 14, wherein the one or more hormones are selected from aldosterone, androstenedione, corticosterone, cortisol, cortisone, 21-deoxycortisol, 11-deoxycortisol, dehydroepiandrosterone (DHEA), 11-deoxycorticosterone, estrone, 17-β-estradiol, estriol, 17-α-hydroxyprogesterone, progesterone, testosterone and dihydrotestosterone.

16. The method of claim 15, wherein one hormone is DHEA, the precursor ion has a mass-to-charge ratio (m/z) of 271 and the product ion has a m/z of 213.

17. The method of claim 15, wherein one hormone is aldosterone, the precursor ion has a m/z of 361 and the product ion has a m/z of 315.2.

18. The method of claim 17, wherein the conditions for tandem mass spectrometry comprise running in positive ion mode.

19. The method of any one of claims 1 to 18, wherein the desolvation line temperature is about 140° C.

20. The method of any one of claims 1 to 19, wherein the interface voltage is from about 2 kV to about 3 kV, optionally about 2 kV or about 3 kV.

21. The method of any one of claims 1 to 20, wherein the liquid chromatography comprises high performance liquid chromatography or ultra-high performance liquid chromatography.

22. The method of claim 21, wherein the liquid chromatography is high performance liquid chromatography.

23. The method of any one of claims 1 to 22, wherein the tandem mass spectrometer is a triple quadrupole mass spectrometer.

24. The method of any one of claims 4 to 23, wherein the sample plate containing the reconstituted samples is loaded onto the LC-MS/MS.

25. The method of any one of claims 4 to 24, wherein the sample plate further comprises one or more calibrators, quality control samples and/or blanks.

26. The method of any one of claims 1 to 25, wherein the method further comprises adding an internal standard to the sample, and, if present, the one or more calibrators, quality control samples and a portion of the blanks.

27. The method of any one of claims 4 to 26, wherein about 10 μL to about 30 μL or about 20 μL of the reconstituted sample is loaded onto the LC-MS/MS.
